(19) United States
(12) Patent Application Publication
(10) Pub. No.: US 2004/0077563 A1
(43) Pub. Date: Apr. 22, 2004

(54) Methods of drug delivery to hepatocytes and treatment of flaviviridae infections

(76) Inventors: Johnson Lau, Newport Beach, CA (US); Zhi Hong, Aliso Viejo, CA (US)

Correspondence Address: Rutan & Tucker, Suite 1400, 611 Anton Boulevard, Costa Mesa, CA, 92626, US

(21) Appl. No.: 10/333,699

(22) PCT Filed: Aug. 21, 2001
(86) PCT No.: PCT/US01/26057

(51) Int. Cl. A61K 31/70

(52) U.S. Cl. 514/42

(57) ABSTRACT

A method of delivering a drug to a hepatocyte includes a step in which a carboxamidine group-containing compound is provided to a hepatocyte having a transporter that transports the compound across the plasma membrane of the hepatocyte, wherein the transport of the compound is substantially not inhibited by ribavirin. Further contemplated methods include a method of inhibiting growth of a virus of a family of flaviviridae in a cell containing system, in which contemplated compounds are presented to a cell in the cell containing system.

This application claims the benefit of U.S. provisional application No. 60/226869, filed Aug. 22, 2000, and U.S. provisional application No. 60/240627, filed Oct. 13, 2000, both of which are incorporated herein by reference.

### FIELD OF THE INVENTION

The field of the invention is pharmaceuticals.

### BACKGROUND OF THE INVENTION

Numerous pharmacologically active molecules are orally or parenterally administered to a system (often a mammal, and typically a human), however, they exhibit their desired activity in intracellular compartments such as the cytoplasm or the nucleus. Consequently, such molecules need to pass across the cellular membrane to reach the site of action. Among various other pharmacologically active molecules, delivery of antiviral drugs into the cytoplasm and/or nucleus is especially important for proper anti-viral activity.

Numerous modifications are known to facilitate transport of such molecules into a target cell. For example, some modifications include portions of or the entire substrate of a cellular transporter (e.g., glucose for GLUT-1, polybasic amino acids for asialoglycoprotein, etc.). Unfortunately, such additions frequently increase the molecular weight, or add an undesirable metabolic load to the pharmacologically active molecules. Depending on the chemical structure of the molecule or type of modification, receptor-mediated uptake can occur through various membrane transporters. For example, various monocarboxylate drugs may be imported by a monocarboxylate transporter, while some endogenous amines and xenobiotics are known to be imported into a cell via an organic cation transporter. Still other transporters import nucleosides in a concentration dependent or energy dependent manner into a cell. A review of various membrane transporters is published by Lee, V. H. (Eur. J. Pharm. Sci. 11, Suppl.2 (2000):41-50), which is incorporated by reference herein.

Other modifications include regio- or organ specific modifications. For example, bile acid adducts may be formed from pharmacologically active molecules to increase the circulation of such molecules in the hepato-biliary system. However, such modifications tend to reduce solubility, and therefore potentially reduce the overall achievable concentration. In another example, modifications on a phosphate group (e.g., formation of a phosphonate ester) convert a drug molecule in a prodrug which has a relatively high organ specificity converted back to the drug by an organ specific enzymatic system (see e.g., U.S. Pat. No. 6,225,460 or U.S. Pat. No. 6,110,903). Still further modifications include antibodies or fragments thereof to increase target specificity of pharmacologically active molecules carrying the antibody. Despite the relatively high selectivity of antibodies, antibodies tend to be problematic with respect to inmunogenicity and their production and/or purification.

Although there are various modifications to pharmacologically active molecules known in the art, all or almost all of them suffer from one or more disadvantages. Therefore, there is still a need to provide improved methods and compositions to increase specificity of pharmacologically active molecules.

### SUMMARY OF THE INVENTION

The present invention is directed to methods of delivering a drug to a hepatocyte and methods of inhibiting growth of flaviridae in a cell containing system.

In one aspect of the inventive subject matter, a method of delivering a drug to a hepatocyte comprises one step, in which an anti-viral or antineoplastic compound having a carboxamidine group is provided. In a further step, it is ascertained that the hepatocyte comprises a transporter that transports the compound across a plasma membrane of the hepatocyte wherein the transport of the compound is substantially not inhibited by ribavirin, and in a still further step, the transporter is presented with the compound.

In another aspect of the inventive subject matter, contemplated compounds comprise a nucleoside with a heterocyclic base coupled to a sugar, wherein the base is preferably a monocyclic base (e.g., 1,2,4-triazole) and the sugar comprises preferably a beta-ribofuranose. Especially preferred compounds include D- and L-Viramidine™.

In a further aspect of the inventive subject matter, the carboxamidine group of contemplated compounds is converted to a carboxamide group in the hepatocyte, and it is further preferred that the compound is enzymatically phosphorylated and thereby accumulates in the hepatocyte. It is especially contemplated that hepatocytes are diseased (e.g., viral infection or infestation). Contemplated transporters specifically include ATP-dependent transporters and transmembrane proteins.

In a still further aspect of the inventive subject matter, a method of inhibiting growth of a virus of a family of flaviviridae in a cell containing system comprises a step in which a pharmaceutical composition having a carboxamidine group is provided. In another step, a cell in the cell containing system is presented with the pharmaceutical composition, wherein the compound is transported across a plasma membrane of the cell, and wherein the transport of the compound is substantially not inhibited by ribavirin.

Various objects, features, aspects and advantages of the present invention will become more apparent from the following detailed description of preferred embodiments of the invention, along with the accompanying drawings.

### BRIEF DESCRIPTION OF THE DRAWINGS

FIG. 1 is a graph depicting nucleoside uptake into different liver cell lines.

FIG. 2 is a graph depicting competition of [³H]-Ribavirin uptake by Ribavirin and Viramidine™ in HepG2 cells.

FIG. 3 is a graph depicting competition of [³H]-Viramidine™ uptake by Ribavirin and Viramidine™ in HepG2 cells.

### DETAILED DESCRIPTION

Studies on transport of 1-beta-D-ribofuranosyl-1,2,4-triazole-3-carboxamide (Ribavirin) into human erythrocytes indicated that Ribavirin enters erythrocytes via a 6-[(4-nitrobenzyl)thio]-9-beta-D-ribofuranosylpurine (NBMPR)-sensitive nucleoside transporter, and it has been demonstrated that transport of Ribavirin into erythrocytes was inhibited by NBMPR at an IC₅₀ of about 0.99 nM (see e.g., Jarvis et al, 1998).

Surprisingly, the inventors discovered that transport of the closely related compound 1-beta-D-ribofuranosyl-1,2,4-triazole-3-carboxamidine (Viramidine™) into hepatocytes appeared to involve a different cellular transporter than the transport of Ribavirin into hepatocytes. The structures of both Ribavirin and Viramidine™ are depicted below in Formula 1 and 2.

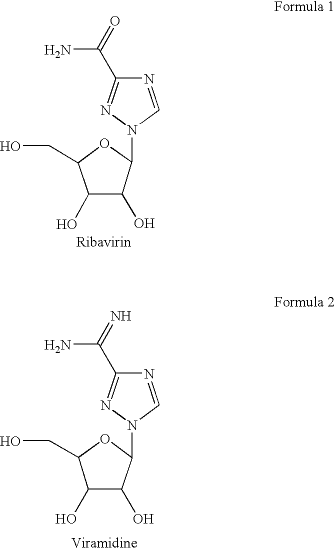

Moreover, the inventors discovered that the rate of uptake into, as well as the overall concentration of Viramidine™ in hepatocytes as compared to Ribavirin was significantly higher, thereby providing a new avenue to increase the effective concentration of an antiviral agent in a hepatocyte.

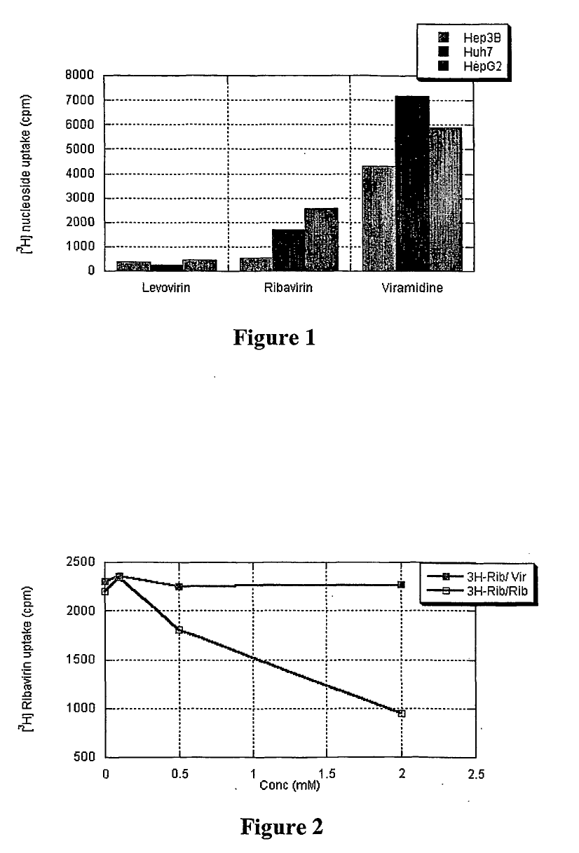

In order to determine whether Ribavirin and Viramidine™ use a similar mode of entry in human hepatocytes, the inventors performed several experiments. First, various strains of hepatocytes (Hep3B, HepG2, and Huh7) were incubated with various nucleotide analogs (e.g., Ribavirin, Levovirin (the L-isomer of Ribavirin), and Viramidine™), and the result of the nucleoside uptake experiments are illustrated in FIG. 1. The test data for the uptake experiment clearly indicate that all three triazole-type nucleoside analogs are taken up into all of the tested hepatocytes, albeit at different rates and/or amounts. More specifically, the D-isomer of Ribavirin was more efficiently taken up over the L-isomer of Ribavirin. More significantly, it became apparent that the carboxamidine-containing nucleoside analog Viramidine™ was taken up to a significantly higher degree in all three cell lines than the carboxamide-containing nucleoside analogs in D- and L-configuration.

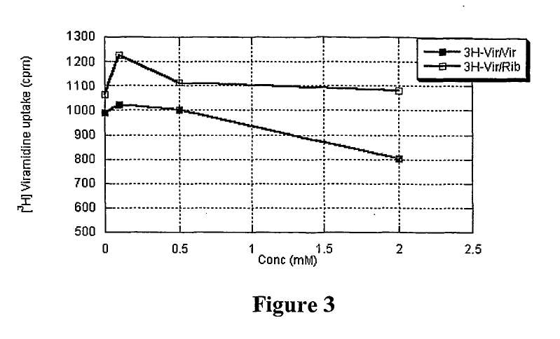

These, and other experiments (data not shown) suggested to the inventors that the uptake of the nucleosides Ribavirin and Viramidine™ could in fact occur via different uptake mechanisms. To address this question, the inventors performed uptake competition experiments in which radiolabeled Ribavirin or Viramidine™ competed for uptake with unlabeled Viramidine™ or Ribavirin, respectively. The result of these experiments is shown in FIGS. 2 and 3. Surprisingly, both experiments showed at least partial competition between Ribavirin and Viramidine™, clearly suggesting differential uptake of Viramidine™ and Ribavirin by hepatocytes.

Consequently, the inventors contemplate that hepatocytes include a cellular uptake and/or transporter system for Viramidine™ or other carboxamidine-containing compounds that is at least partially, if not entirely different from the transporter system for Ribavirin (i.e., the NBMPR-sensitive nucleoside transporter). Thus, it is contemplated that hepatocytes can be targeted with pharmacological molecules that selectively utilize the Viramidine™ uptake and/or transporter system. The term “transporter system” and “transporter” are used interchangeably herein and refer to a membrane associated (transmembrane protein, or protein located/anchored in the outer or inner cell membrane) protein or protein complex that facilitates influx of a compound across a plasma membrane into a cell. Typically, such transporters will be energy dependent (e.g., ATP dependent), and contemplated transporters may further be regulated by various factors (e.g., membrane potential, Ca²⁺ concentration, cGTP, etc). Moreover, it is contemplated that suitable transporter systems may be inhibited by a second compound having a carboxamidine group.

With respect to contemplated pharmacological molecules, it should be appreciated that suitable molecules not only include Viramidine™, but also numerous modifications of Viramidine™. For example, particularly contemplated modifications include the L-isomer of Viramidine™. Further contemplated modifications especially include mono-, di-, and triphosphate forms of Viramidine™, and all chemically and/or physiologically reasonable prodrug forms. Examples for such prodrug forms are set forth in PCT application PCT/US01/08713 with the title “Nucleoside Compounds and Uses Thereof”, filed Mar. 15, 2001, which is incorporated by reference herein.

Moreover, it is contemplated that further appropriate nucleosides and nucleoside analogs include nucleotides and nucleotide analogs that include a carboxamidine function, or a modified carboxamidine function, wherein the term “modified carboxamidine function” particularly includes groups having the following structure:

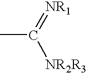

wherein R₁, R₂, and R₃ independently include H, alkyl, alkenyl, alkynyl, aryl, and alkaryl, all of which may be linear or branched, and further include functional groups. Particularly preferred functional groups include polar, basic, acidic, electrophilic and nucleophilic groups (carboxylate, thiol, tertiary or quarternary ammonium groups, esters, hydroxyls, amides, imides, ethers, etc.), and halogens. In further alternative aspects, it is contemplated that the bases of suitable nucleoside or nucleotide analogs includes naturally occurring as well as non-naturally occurring bases, and especially contemplated alternative bases are guanine, hypoxanthine, uracil, cytidine, adenine, cytidine, fluorouracil, monocyclic bases (e.g., 1,2,4-triazole), etc.

Similarly, the sugar moiety may include natural and/or modified sugars. For example, where natural sugars are preferred, non-ribofuranose sugars such as arabinose may be incorporated. On the other hand, non-natural sugars may advantageously include conformationally constrained or locked sugars, sugars with non-OH substituents (e.g., ethynyl, halogen, alkyl), or deoxy-sugars. Particularly preferred nucleosides will exhibit an anti-viral activity against flaviviridae, and especially against hepatitis C-type viruses (e.g., HCV virus or pestivirus), or anti-neoplastic activity against neoplastic hepatocytes.

It should further be appreciated that numerous cells other than hepatocytes (which may or may not be neoplastic cells) are also contemplated, and suitable cells will generally include all cells that exhibit Viramidine™ uptake that is substantially unaffected by competing Ribavirin in a concentration range as employed in the experiments of FIGS. 2 and 3. The term substantially unaffected refers to a reduction of Viraindine™ uptake of no more than 15%, preferably no more than 10%, more preferably no more than 8%, and most preferably no more than 5%. However, it is generally contemplated that hepatocytes, and especially diseased hepatocytes (e.g., viral infection or infestation) are preferred cells.

Thus, it should be appreciated that toxicity of a nucleoside or nucleotide compound can be reduced by modifying the compound to include a carboxamidine moiety or modified carboxamidine moiety as described above, wherein target cells for modified compounds include a transporter system, which non-target cells lack. For example, Ribavirin is readily taken up by erythrocytes and hepatocytes, and retained after intracellular phosphorylation, frequently leading to hemolytic anemia in patients when the patients are under a high-dose or long-term administration regimen of Ribavirin. Interestingly, however, Viramidine™ is not taken up by erythrocytes, while still being taken up by hepatocytes. It is contemplated that this difference in uptake is attributable to the lack of a Viramidine™ uptake and/or transporter system. In fact, experiments show that Viramidine™ is not taken up into erythrocytes (data not shown) to any appreciable degree.

Moreover, due to the relatively high deaminase/deamidase activity in hepatocytes, Viramidine™ is converted in the liver to Ribavirin (unpublished results), and subsequently intracellularly phosphorylated to Ribavirinmonophosphate, which is typically retained in the hepatocytes. Therefore, it is contemplated that by including a carboxamidine group to a molecule and exploiting the relatively high deaminase/deamidase acitivity of hepatocytes, molecules may be specifically targeted to the liver. With respect to contemplated molecules having a —C(NR₁)(NR₂R₃) it should be appreciated that various deaminase/deamidase acitivities of hepatocytes are sufficiently high to convert such compounds from a prodrug form into an active drug form (e.g., drug activity is increased when carboxamide group is present).

Without wishing to be bound to a particular theory or enzymatic mechanism, it is contemplated that the conversion of a carboxamidine-containing compound to the corresponding carboxamide-containing compound may include an enzymatic conversion, and that the enzyme converting a carboxamidine-containing compound to the corresponding carboxamide-containing compound (e.g., converting Viramidine™ to Ribavirin) belongs to the class of aminohydrolases. Particularly contemplated aminohydrolases include adenosine-or cytosine-deaminases, aryl-deamidase, and glutamate-pyruvate transaminase.

Consequently, the inventors contemplate that a method of delivering a drug to a hepatocyte comprises one step in which an anti-viral or anti-neoplastic compound having a carboxamidine group is provided. In another step, it is ascertained that the hepatocyte comprises a transporter that transports the compound across a plasma membrane of the hepatocyte wherein the transport of the compound is substantially not inhibited by ribavirin. The term “substantially not inhibited by ribavirin” refers to an inhibition of no more than 20%, more typically no more than 15%, even more typically no more than 10%, and most typically no more than 8%, wherein the concentration of Ribavirin is between 100 μM and 2 mM under test conditions as described in the experimental section. In a still further step, the transporter is presented with the compound.

In a further aspect of the inventive subject matter, the inventors discovered that growth of a particular family of viruses, more specifically the flaviviridae, can be inhibited in a cell containing medium by adding some of the contemplated compounds, and especially the hydrochloride salt of Viramidine™ (1-beta-D-ribofuranosyl-1,2,4-triazole-3-carboxamidine*HCl) to a cell-containing medium (data not shown). The family of flaviviridae has recently gained special attention due to several occurrences of West Nile Virus encephalitis in the eastern part of the USA, resulting in at least 7 reported deaths and at least 62 cases of severe disease. Moreover, hepatitis C-like viruses (and the HCV virus) also belong to the family of flaviviridae, further adding to the significance of flaviviridae with respect to public and private health. While not wishing to be bound by a particular theory or hypothesis, it is contemplated that increased transport of Viramidine™*HCl is particularly beneficial for treatment of flaviridae-virus infections.

Consequently, infections particularly contemplated to be treated with Viramidine™*HCl include hepatitis C virus (HCV), West Nile virus, yellow fever virus, Kokobera virus, Murray Valley encephalitis, Kunjun virus, and Langat virus, and viruses from the Tick-borne encephalitis virus group, the Japanese encephalitis group, and the Dengue Group.

Therefore, a method of inhibiting growth of a virus belonging to a family of flaviviridae in a cell containing system will include a step in which a pharmaceutical composition having a carboxamidine group is provided. In another step, a cell in the cell containing system is presented with the pharmaceutical composition, wherein the compound is transported across a plasma membrane of the cell, wherein the transport of the compound is substantially not inhibited by ribavirin. Particularly preferred cell containing systems include in vitro (e.g., petri dish or cell culture) and in vivo systems (e.g., mammals, and especially human), and it is further especially contemplated that suitable cells include a hepatocyte that is infected with the virus.

It is contemplated that Viramidine™*HCl (in either D- or L-configuration) will be administered in any appropriate pharmaceutical formulation, and under any appropriate protocol. Thus, administration may take place orally, parenterally (including subcutaneous injections, intravenous, intramuscularly, by intrasternal injection or infusion techniques), by inhalation spray, or rectally, topically and so forth, and in dosage unit formulations containing conventional non-toxic pharmaceutically acceptable carriers, adjuvants and vehicles. By way of example, it is contemplated that Viramidine™*HCl can be formulated in admixture with a pharmaceutically acceptable carrier. For example, Viramidine™*HCl can be administered orally as pharmacologically acceptable salts. Because Viramidine™*HCl is mostly water soluble, it can be administered intravenously in physiological saline solution (e.g., buffered to a pH of about 7.2 to 7.5). Conventional buffers such as phosphates, bicarbonates or citrates can be used for this purpose. Of course, one of ordinary skill in the art may modify the formulations within the teachings of the specification to provide numerous formulations for a particular route of administration without rendering Viramidine™*HCl unstable or compromising their therapeutic activity. In particular, the modification of Viramidine™*HCl to render it more soluble in water or another vehicle, for example, may be easily accomplished by minor modifications (salt formulation, esterification, etc.) that are well within the ordinary skill in the art. It is also well within the ordinary skill of the art to modify the route of administration and dosage regimen of a particular compound in order to manage the pharmacokinetics of the present compounds for maximum beneficial effect in patients.

In certain pharmaceutical dosage forms, the pro-drug form of the compounds, especially including acylated (acetylated or other) derivatives, pyridine esters and various salt forms of the present compounds are preferred. One of ordinary skill in the art will recognize how to readily modify the present compounds to pro-drug forms to facilitate delivery of active compounds to a target site within the host organism or patient. One of ordinary skill in the art will also take advantage of favorable pharmacokinetic parameters of the pro-drug forms, where applicable, in delivering the present compounds to a targeted site within the host organism or patient to maximize the intended effect of the compound.

In addition, Viramidine™*HCl may be administered alone or in combination with other agents for the treatment of the above infections or conditions. Combination therapies according to the present invention comprise the administration of at least one compound of the present invention or a functional derivative thereof and at least one other pharmaceutically active ingredient. The active ingredient(s) and pharmaceutically active agents may be administered separately or together and when administered separately this may occur simultaneously or separately in any order. The amounts of the active ingredient(s) and pharmaceutically active agent(s) and the relative timings of administration will be selected in order to achieve the desired combined therapeutic effect. Preferably, the combination therapy involves the administration of Viramidine™*HCl or a physiologically functional derivative thereof and one of the agents mentioned herein below.

Examples of other drugs or active ingredients contemplated to be effective in combination with Viramidine™ are anti-viral agents such as various interferons, including but not limited to interferon α and γ, Ribavirin, acyclovir, and AZT™; anti-fungal agents such as tolnaftate, Fungizone™, Lotrimin™, Mycelex™, Nystatin and Amphoteracin; anti-parasitics such as Mintezol™, Niclocide™, Vermox™, and Flagyl™, bowel agents such as Immodium™, Lomotil™ and Phazyme™; anti-tumor agents such as interferon α and γ, Adriarnycin™, Cytoxan™, Imuran™, Methotrexate, Mithracin™, Tiazofurin™, Taxol™; dermatologic agents such as Aclovate™, Cyclocort™, Denorex™, Florone™, Oxsoralen™, coal tar and salicylic acid; migraine preparations such as ergotamine compounds; steroids and immunosuppresants not listed above, including cyclosporins, Diprosone™, hydrocortisone; Floron™, Lidex™, Topicort and Valisone; and metabolic agents such as insulin, and other drugs which may not nicely fit into the above categories, including cytolines such as IL2, IL4, IL6, IL8, IL10 and IL12. Especially preferred primary drugs are AZT, 3TC, 8-substituted guanosine analogs, 2,3-dideoxynucleosides, interleukin II, interferons such as IαB-interferons, tucaresol, levamisole, isoprinosine and cyclolignans.

Examples of such further therapeutic agents include agents that are effective for the modulation of immune systems or associated conditions such as AZT, 3TC, 8-substituted guanosine analogs, 2′,3′-dideoxynucleosides, interleukin II, interferons, such as α-interferon, tucaresol, levamisole, isoprinosine and cyclolignans. Certain compounds according to the present invention may be effective for enhancing the biological activity of certain agents according to the present invention by reducing the metabolism or inactivation of other compounds and as such, are co-administered for this intended effect.

It is contemplated that various alternative dosages are also appropriate, including dosages between 1-200 mg/day and less. In preferred embodiments, appropriate dosages range from 10-200 mg/day. In more preferred embodiments, dosages range from 50-200 mg/day, and in even more preferred embodiments, dosages range from 5-100 mg/day. In general, the appropriate dosage will depend on multiple parameters, including the type of virus infection, the stage of the virus infection, the desired plasma concentration of Viramidine™, the duration of the treatment, etc. For example, while treatment success may be achieved with some viral infections at relatively low plasma concentrations of Viramidine™, other viral infections may require relatively high dosages.

Administration of the active compound may range from continuous (intravenous drip) to several oral administrations per day (for example, Q.I.D.) and may include oral, topical, parenteral, intramuscular, intravenous, sub-cutaneous, transdermal (which may include a penetration enhancement agent), buccal and suppository administration, among other routes of administration.

To prepare the pharmaceutical compositions according to the present invention, a therapeutically effective amount of one or more of the compounds according of the present invention is preferably intimately admixed with a pharmaceutically acceptable carrier according to conventional pharmaceutical compounding techniques to produce a dose. A carrier may take a wide variety of forms depending on the form of preparation desired for administration, e.g., oral or parenteral. In preparing pharmaceutical compositions in oral dosage form, any of the usual pharmaceutical media may be used. Thus, for liquid oral preparations such as suspensions, elixirs and solutions, suitable carriers and additives including water, glycols, oils, alcohols, flavoring agents, preservatives, coloring agents and the like may be used. For solid oral preparations such as powders, tablets, capsules, and for solid preparations such as suppositories, suitable carriers and additives including starches, sugar carrier, such as dextrose, mannitol, lactose and related carriers, diluents, granulating agents, lubricants, binders, disintegrating agents and the like may be used. If desired, the tablets or capsules may be enteric-coated or sustained release by standard techniques.

For parenteral formulations, the carrier will usually comprise sterile water or aqueous sodium chloride solution, though other ingredients including those that aid dispersion may be included. Of course, where sterile water is to be used and maintained as sterile, the compositions and carriers must also be sterilized. Injectable suspensions may also be prepared, in which case appropriate liquid carriers, suspending agents and the like may be employed.

### EXAMPLES

### Uptake of Ribavirin, Viramidine™, and Levovirin™ by Different Human Liver Cell Lines

Hep3B, HepG2 & Huh7 cell lines were used at a concentration of 50,000 cells/well in a 24-wells plate, and incubated with [³H]-labeled Ribavirin, Levovirin™, and Viramidine™ (20 μCi/ml) plus 10 μM cold nucleoside for 4 hr at 37° C. The cells were washed with PBS and lysed, and the radioactivity in the cell lysate (i.e.the amount of labeled nucleoside taken up by the cells) was determined in a scintillation counter.

### Nucleoside Competition Assay

50,000 HepG2 cells/well were placed in a 24-wells plate and incubated with [3H]-labeled nucleoside (20 μCi/ml). Competition was performed with increasing concentrations of cold (0, 100 μM, 500 μM, and 2 mM) competitor nucleoside. Incubation time was 2 hrs at 37° C. The cells were then washed with PBS and lysed. The radioactivity in the cell lysate (i.e. the amount of labeled nucleoside taken up by the cells) was determined in a scintillation counter.

Thus, specific embodiments of methods of drug delivery to hepatocytes and treatment of flaviviridae infections have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

1. A method of delivering a drug to a hepatocyte, comprising:
providing a compound having a carboxamidine group wherein the compound has an activity selected from the group consisting of anti-viral activity and anti-neoplastic activity;
ascertaining that the hepatocyte comprises a transporter that transports the compound across a plasma membrane of the hepatocyte wherein the transport of the compound is substantially not inhibited by ribavirin; and
presenting the transporter with the compound.

2. The method of claim 1, wherein the compound comprises a nucleoside having a base coupled to a sugar.

3. The method of claim 2, wherein the base comprises a monocyclic base.

4. The method of claim 3, wherein the monocyclic base comprises a 1,2,4-triazole.

5. The method of claim 2, wherein the sugar comprises a β-ribofuranose.

6. The method of claim 2, wherein the nucleoside comprises 1-beta-D-ribofuranosyl-1,2,4-triazole-3-carboxamidine.

7. The method of claim 2, wherein the nucleoside comprises 1-beta-L-ribofuranosyl-1,2,4-triazole-3-carboxamidine.

8. The method of claim 1, wherein the carboxamidine group is converted to a carboxamide group in the hepatocyte.

9. The method of claim 8, wherein the carboxamide group is enzymatically phosphorylated and thereby accumulates in the hepatocyte.

10. The method of claim 1, wherein the anti-viral activity is an anti-viral activity against flaviviridae.

11. The method of claim 10, wherein the anti-viral activity against flaviviridae is an anti-viral activity against a pestivirus and a hepa C virus.

12. The method of claim 1, wherein the anti-neoplastic activity includes an anti-neoplastic activity against neoplastic hepatocytes.

13. The method of claim 1, wherein the transporter comprises an ATP-dependent transporter.

14. The method of claim 1, wherein the transporter comprises a transmembrane protein.

15. The method of claim 1, wherein the hepatocyte is diseased.

16. The method of claim 1, wherein transport of the compound comprises transport that is inhibited by a second compound having a carboxamidine group.

17. A method of inhibiting replication of a virus belonging to a family of flaviviridae in a cell containing system comprising:
providing a pharmaceutical composition having a carboxamidine group; and
presenting a cell in the cell containing system with the pharmaceutical composition, wherein the compound is transported across a plasma membrane of the cell, wherein the transport of the compound is substantially not inhibited by ribavirin.

18. The method of claim 17, wherein the virus is a West Nile virus.

19. The method of claim 17, wherein the virus is a hepatitis C virus.

20. The method of claim 17, wherein the cell containing system is an in vitro system.

21. The method of claim 17, wherein the cell containing system is an in vivo system.

22. The method of claim 22, wherein the in vivo system has at least one hepatocyte that is infected with the virus.

23. The method of claim 17, wherein the pharmaceutical compound comprises 1-beta-D-ribofuranosyl-1,2,4-triazole-3-carboxamidine*HCl

24. The method of claim 17, wherein the pharmaceutical compound is in L-configuration.
